(19) United States
(12) Patent Application Publication
(10) Pub. No.: US 2009/0232919 A1
(43) Pub. Date: Sep. 17, 2009

(54) Composition Comprising Liquiritigenin for Preventing and Treating Liver Disease

(76) Inventors: Sang Geon Kim, Seoul (KR); Sang Chan Kim, Deagu (KR); Young Woo Kim, Busan (KR)

Correspondence Address: MEYERTONS, HOOD, KIVLIN, KOWERT & GOETZEL, P.C., P.O. BOX 398, AUSTIN, TX, 78767-0398, US

(21) Appl. No.: 12/087,038

(22) PCT Filed: Jan. 5, 2007
(86) PCT No.: PCT/KR2007/000081

§ 371 Date: Jun. 24, 2008

(30) Foreign Application Priority Data
Jan. 9, 2006 (KR) 10-2006-0002253

(51) Int. Cl. A61K 36/48; A61K 31/352; A61P 1/16; C07D 311/30

(52) U.S. Cl. 424/757; 514/456; 549/403

(57) ABSTRACT

The present invention is related to a composition comprising an extract of licorice or liquiritigenin isolated therefrom significantly decrease the blood concentration of LDH and ALT enzyme, central necrosis and inflammation in acetaminophen-induced hepato-toxicity animal model when the inventive extract or compound of the present invention was orally and intravenously administrated thereto. Accordingly, the inventive compositions according to the present invention are useful in the prevention and treatment of the liver diseases and can be used as safe and efficient hepato-protective

### TECHNICAL FIELD

The present invention is related to a composition comprising a licorice root extract and liquiritigenin isolated therefrom as an effective ingredient for the prevention and treatment of liver diseases and a use thereby.

### BACKGROUND ART

Liver disorders are one of the most frequently occurring diseases in present human being exposed by various unfavorable environments for example, polluting substance, toxic substance such as overdrinking, smoke etc as well as psychological stress, which could be recovered by rest however it could be severed to give rise to other disease such as the disorder of immune system. There have been reported that toxic substance such as acetoaminophen, carbon tetrachloride, D-galactosamine etc. cause to toxic in liver, especially, acetoaminophen has been frequently used as a standard hepato-toxic indicator evaluating the treating an protecting efficacy of new drug or new agent (D. J. Jollow et al., J. Pharmacol. Exp. Ther. 187, pp 195-202, 1973).

Over-dosing of acetaminophen causes to liver injury characterized by secondary liver injury such as extensive necrosis of liver cells whereas effective amount of acetoaminophen shows potent anti-inflammatory and anti-pyretic agent (B. H. Rumack, Hepatology 40, pp 10-15, 2004). Such toxicity of acetoaminophen is caused by the metabolized actoaminophen form by the action of cytochrome P450 in liver, i.e., NAPQ1 (N-acetyl-p-benzoquinone imine). It could be effectively detoxified by GSH (glutathione) when an effective amount of it was administrated however it could not be excreted from human body when over dose of it was administrated resulting in toxic effect on liver organ (J. D. Gibson et al., Chem. Res. Toxicol., 9, pp 580-585 1996) characterized by hepatic central necrosis, degeneration of hepatic cell, and occurrence of inflammatory cells (D. Zakin et al., Hepatology, pp 759-762, 1990). The concentration of human GSH maintains regularly however it abruptly drops under specific abnormal condition, which causes to increase the susceptibility against outer toxic substance (E. Y park et al., Chem. Biol. Interact. 155, pp 82-96, 2005).

NAC (N-acetylcysteine) has been known as a sole treating agent to treat the toxicity caused by acetoaminophen until now. There still remains to develop more effective agent to treat the toxicity caused by acetoaminophen.

Licorice root, a root of Glycyrrhiza uralensis FISCH, Glycyrrhiza glabra L. and the like belongs to Leguminosae has been reported to contain triterpene saponin such as glycyrrhizin and several flavonids such as liquiritigenin, liquiritin, neoliquiritin, neoisoliquiritin etc, which has been used to treat cough, gastric ulcer, hypertension, etc.

However, there has been not reported or disclosed about the therapeutic effect of licorice extract or liquiritigenin on liver disease caused by acetaminophen in any of above cited literatures, the disclosures of which are incorporated herein by reference.

Therefore, the present inventors have endeavored to find the effective agent for enhancing hepato-protective efficacy and to study the pharmacological effect of liquiritigenin through various in vitro and animal model tests and finally, the present inventors have found that licorice extract or liquiritigenin is effective in treating and preventing liver diseases as a hepato-protective agent.

### DISCLOSURE OF INVENTION

### Technical Problem

According to one aspect, the present invention provides a pharmaceutical composition comprising the extract of licorice or liquiritigenin isolated therefrom as an active ingredient for preventing and treating liver diseases.

The present invention also provides a method for treating liver disease by protecting hepatic cell in a mammal comprising administering to said mammal an effective amount of above-mentioned extract or the compound isolated therefrom, together with a pharmaceutically acceptable carrier thereof.

The present invention also provides a use of the above described extract or the compound isolated therefrom for the preparation of for manufacture of medicament employed for treating or preventing liver disease in human or mammal.

The present invention also provides a health functional food comprising the above-described extract or the compound isolated therefrom for the prevention or Improvement of liver disease by protecting hepatic cell as an active ingredient in an amount effective to preventing and improving liver disease, together with a sitologically acceptable additive.

### Technical Solution

Accordingly, it is an object of the present invention to provide a pharmaceutical composition comprising the extract of licorice or liquiritigenin isolated therefrom as an active ingredient in an amount effective to preventing and treating liver disease, together with a pharmaceutically acceptable carrier.

It is another object of the present invention to provide a use of extract of licorice or liquiritigenin isolated therefrom for manufacture of medicament employed for treating or preventing liver disease in human or mammal.

It is the other object of the present invention to provide a method for treating liver diseases by protecting hepatic cell in a mammal comprising administering to said mammal an effective amount of extract of licorice or liquiritigenin isolated therefrom, together with a pharmaceutically acceptable carrier thereof.

The extract of licorice disclosed herein comprise the extract of glycyrrhiza uralensis, glycyrrhiza glabra L. or the like, preferably, glycyrrhiza uralensis, and the extract can be obtained by extracting with distilled water, lower alcohols such as methanol, ethanol and the like, or the mixtures thereof, preferably, methanol, more preferably, liquiritigenin-abundant extract of licorice, for example, which can be prepared by the procedure consisting of the steps: purifying the extract of licorice with repeated column chromatographic method to obtain purified liquiritin fraction; treating to acidic hydrolysis and neutralizing the pH of the solution with alkali; and subjecting column chromatography to obtain the purposed liquiritigenin-abundant extract of the present invention.

It is the other object of the present invention to provide a pharmaceutical composition comprising the combination of the extract of licorice or liquiritigenin isolated therefrom and DDB (dimethyl-4,4′-dimethoxy-5,6,5′,6′-dimethylene dioxybiphenyl-2,2′-dicarboxylate) as an active ingredient in an amount effective to preventing and treating liver disease, together with a pharmaceutically acceptable carrier.

It is another object of the present invention to provide a use of the combination of the extract of licorice or liquiritigenin isolated therefrom and DDB for manufacture of medicament employed for treating or preventing liver disease in human or mammal.

It is the other object of the present invention to provide a method for treating liver disease by protecting hepatic cell in a mammal comprising administering to said mammal an effective amount of the combination of the extract of licorice or liquiritigenin isolated therefrom and DDB, together with a pharmaceutically acceptable carrier thereof.

In accordance with one aspect of the present invention, there provided a health functional food comprising the extract of licorice or liquiritigenin isolated therefrom for the prevention or improvement of liver disease by protecting hepatic cell as an active ingredient in an amount effective to prevent and improve liver disease, together with a sitologically acceptable additive.

In accordance with one aspect of the present invention, there provided a health functional food comprising the combination of the extract of licorice or liquiritigenin isolated therefrom and DDB for the prevention or improvement of liver disease by protecting hepatic cell as an active ingredient in an amount effective to prevent and improve liver disease, together with a sitologically acceptable additive.

The liver disease disclosed herein comprises acute or chronic hepatitis, hepatomegaly, hepatophyma, hepatocirrhosis and liver cancer, preferably, acute or chronic hepatitis and hepatocirrhosis.

The herb, which can be used in the present invention, but not intent to limit thereto, include the same genus plants which would be apparent to those skilled in the art and have be used for identical or similar purpose and can be substituted for the prevention and treatment of liver disease.

The pharmaceutical composition for treating liver diseases could contain about 0.01 to 95 w/w %, preferably 0.5 to 50 w/w % of inventive extract or compound of present invention based on the total weight of the composition.

An inventive extract and compound may be prepared in accordance with the following preferred embodiment.

For the present invention, the above-described extract of licorice or liquiritigenin isolated therefrom can be prepared by following procedure;

For example, the licorice, for example, i.e., glycyrrhiza uralensis, is washed, dried, and mixed with 1 to 20-fold, preferably, 5 to 15-fold volume of distilled water, alcohols such as methanol, ethanol and the like, or the mixtures thereof, preferably, methanol; the solution is enfleuraged at the temperature ranging from 0 to room temperature, preferably room temperature, for the period ranging from 12 hours to 1 week, preferably 48 to 72 hours or heated with reflux extraction at the temperature ranging from 80 to 120° C., preferably above 105° C., for the period ranging from 1 to 24 hours, preferably 2 to 5 hours with 2 to 5 times, or extracted by sonication, reflux or conventional extraction; the solution is filtered to obtain the crude extract of licorice of the present invention.

To obtain more preferable liquiritigenin-abundant extract of the present invention, the crude extract of licorice prepared from the above step is subjected to repeated column chromatography eluted with mixed solvent system to obtain liquiritin-abundant extract of licorice; the purified extract is subjected to acid hydrolysis using by strong acid such as HCl to remove the sugar-moiety of liquiritin; the reactant is neutralized with alkali solution such as NaOH; and the solution is subjected to Silica gel column chromatography eluting with mixture solvent system (CHCl₃ and acetone) to obtain liquiritigenin-abundant extract of the present invention.

To obtain pure liquiritigenin of the present invention, the liquiritigenin-abundant extract is subjected to further purification process such as Silicagel column chromatography or re-crystallization method to obtain liquiritigenin of the present invention.

To obtain the combination of the extract of licorice or liquiritigenin isolated therefrom and DDB of the present invention, the extract of licorice and liquiritigenin prepared by the above method may be mixed with DDB with mixed ratio ranging from 1-20:1 to 1:10 (w/w %), preferably, 1-10:1-5 (w/w %), more preferably, 1:1-2 (w/w %) to obtain the combined composition of the present invention.

It is another object of the present invention to provide a process for preparing the above-described extract of licorice and liquiritigenin isolated therefrom as described above for the preparation of composition effective in treating or preventing liver disease.

It is the other object of the present invention to provide a method for preparing liquiritigenin-abundant extract from the extract of licorice comprising the steps consisting of, washing, drying licorice; mixing with 10 to 15-fold volume of distilled water, alcohols such as methanol, ethanol and the like, or the mixtures thereof, enfleuraging the solution at the temperature ranging from 0 to room temperature, 48 to 72 hours; filtering the residue to obtain the crude extract of licorice; subjecting the extract to repeated column chromatography eluted with mixed solvent system to obtain liquiritin-abundant extract of licorice; subjecting the extract to acid hydrolysis using by strong acid to remove the sugar-moiety of liquiritin; neutralizing the solution with alkali solution; subjecting the solution to Silica gel column chromatography eluting with mixture solvent system (CHCl₃ and acetone) to obtain liquiritigenin-abundant extract of the present invention.

The inventive compound can be transformed into their pharmaceutically acceptable salt and solvates by the conventional method well known in the art. For the salts, acid-addition salt thereof formed by a pharmaceutically acceptable free acid thereof is useful and can be prepared by the conventional method. For example, after dissolving the compound in the excess amount of acid solution, the salts are precipitated by the water-miscible organic solvent such as methanol, ethanol, acetone or acetonitrile to prepare acid addition salt thereof and further the mixture of equivalent amount of compound and diluted acid with water or alcohol such as glycol monomethylether, can be heated and subsequently dried by evaporation or filtrated under reduced pressure to obtain dried salt form thereof.

As a free acid of above-described method, organic acid or inorganic acid can be used. For example, organic acid such as methansulfonic acid, p-toluensulfonic acid, acetic acid, trifluoroacetic acid, citric acid, maleic acid, succinic acid, oxalic acid, benzoic acid, lactic acid, glycolic acid, gluconic acid, galacturonic acid, glutamic acid, glutaric acid, glucuronic acid, aspartic acid, ascorbic acid, carbonylic acid, vanillic acid, hydroiodic acid and the like, and inorganic acid such as hydrochloric acid, phosphoric acid, sulfuric acid, nitric acid, tartaric acid and the like can be used herein.

Further, the pharmaceutically acceptable metal salt form of inventive compound may be prepared by using base. The alkali metal or alkali-earth metal salt thereof can be prepared by the conventional method, for example, after dissolving the compound in the excess amount of alkali metal hydroxide or alkali-earth metal hydroxide solution, the insoluble salts are filtered and remaining filtrate is subjected to evaporation and drying to obtain the metal salt thereof. As a metal salt of the present invention, sodium, potassium or calcium salt are pharmaceutically suitable and the corresponding silver salt can be prepared by reacting alkali metal salt or alkali-earth metal salt with suitable silver salt such as silver nitrate.

The pharmaceutically acceptable salt of the compound comprise all the acidic or basic salt which may be present at the compounds, if it does not indicated specifically herein. For example, the pharmaceutically acceptable salt of the present invention comprise the salt of hydroxyl group such as the sodium, calcium and potassium salt thereof; the salt of amino group such as the hydrogen bromide salt, sulfuric acid salt, hydrogen sulfuric acid salt, phosphate salt, hydrogen phosphate salt, dihydrophosphate salt, acetate salt, succinate salt, citrate salt, tartarate salt, lactate salt, mandelate salt, methanesulfonate(mesylate) salt and p-toluenesulfonate (tosylate) salt etc, which can be prepared by the conventional method well known in the art.

It is still another object of the present invention to provide a pharmaceutical composition comprising the pulverized form, extracted form or dried extract form of above crude drug extract obtained by above described process as an active ingredient for preventing and treating liver disease.

The inventive composition of the present invention prepared by above-described process significantly decreases blood concentration of LDH and ALT enzyme, central necrosis and inflammation in acetaminophen-induced hepato-toxicity animal model when the inventive extract or compound of the present invention was orally and intravenously administrated thereto. When the oral acute toxicity of the extract was tested, the extract had no apparent effect on mortality, clinical signs, body weight changes, and gross findings at necropsy.

The pharmaceutical composition for treating liver diseases could contain about 0.01 to 99.9 w/w %, preferably 0.1 to 90 w/w % of the above crude drug composition of present invention based on the total weight of the composition.

The inventive composition may additionally comprise conventional carrier, adjuvants or diluents in accordance with a using method. It is preferable that said carrier is used as appropriate substance according to the usage and application method, but it is not limited. Appropriate diluents are listed in the written text of Remington's Pharmaceutical Science (Mack Publishing co, Easton Pa.).

Hereinafter, the following formulation methods and excipients are merely exemplary and in no way limit the invention.

The composition according to the present invention can be provided as a pharmaceutical composition containing pharmaceutically acceptable carriers, adjuvants or diluents, e.g., lactose, dextrose, sucrose, sorbitol, mannitol, xylitol, erythritol, maltitol, starches, acacia rubber, alginate, gelatin, calcium phosphate, calcium silicate, cellulose, methyl cellulose, polyvinyl pyrrolidone, water, methylhydroxy benzoate, propylhydroxy benzoate, talc, magnesium stearate and mineral oil. The formulations may additionally include fillers, anti-agglutinating agents, lubricating agents, wetting agents, flavoring agents, emulsifiers, preservatives and the like. The compositions of the invention may be formulated so as to provide quick, sustained or delayed release of the active ingredient after their administration to a patient by employing any of the procedures well known in the art.

For example, the compositions of the present invention can be dissolved in oils, propylene glycol or other solvents which are commonly used to produce an injection. Suitable examples of the carriers include physiological saline, polyethylene glycol, ethanol, vegetable oils, isopropyl myristate, etc., but are not limited to them. For topical administration, the compounds of the present invention can be formulated in the form of ointments and creams.

Pharmaceutical formulations containing inventive composition may be prepared in any form, such as oral dosage form (powder, tablet, capsule, soft capsule, aqueous medicine, syrup, elixirs pill, powder, sachet, granule), or topical preparation (cream, ointment, lotion, gel, balm, patch, paste, spray solution, aerosol and the like), suppository, or sterile injectable preparation (solution, suspension, emulsion).

The inventive composition of the present invention in pharmaceutical dosage forms may be used in the form of their pharmaceutically acceptable salts, and also may be used alone or in appropriate association, as well as in combination with other pharmaceutically active compounds.

The desirable dose of the inventive composition varies depending on the condition and the weight of the subject, severity, drug form, route and period of administration, and may be chosen by those skilled in the art. However, in order to obtain desirable effects, it is generally recommended to administer at the amount ranging 0.01-10 g/kg, preferably, 1 to 5 g/kg by weight/day of the inventive composition of the present invention. The dose may be administered in a single or multiple doses per day. In terms of composition, the crude drug composition should be present between 0.01 to 80% by weight, preferably 0.5 to 50% by weight based on the total weight of the composition.

The pharmaceutical composition of present invention can be administered to a subject animal such as mammals (rat, mouse, domestic animals or human) via various routes. All modes of administration are contemplated, for example, administration can be made orally, rectally or by intravenous, intramuscular, subcutaneous, intracutaneous, intrathecal, epidural or intracerebroventricular injection.

In accordance with one aspect of the present invention, there provided a health functional food comprising the above extract or the compound for the prevention or improvement of liver disease by protecting hepatic cell as an active ingredient in an amount effective to preventing and improving liver disease, together with a sitologically acceptable additive.

The crude drug composition of inventive health functional food is used in the form of pulverized form thereof, extracted form therefrom or dried extract form thereof.

The health functional food composition for preventing and improving liver disease could contain about 0.01 to 95 w/w %, preferably 0.5 to 80 w/w % of the above inventive composition of present invention based on the total weight of the composition.

Above described composition therein can be added to food, additive or beverage for prevention and improvement of liver diseases. For the purpose of preventing and improving liver diseases, wherein, the amount of above described crude drug composition in food or beverage may generally range from about 0.1 to 15 w/w %, preferably 1 to 10 w/w % of total weight of food for the health food composition and 1 to 30 g, preferably 3 to 10 g on the ratio of 100 ml of the health beverage composition.

Providing that the health beverage composition of present invention contains above described crude drug composition as an essential component in the indicated ratio, there is no particular limitation on the other liquid component, wherein the other component can be various deodorant or natural carbohydrate etc such as conventional beverage. Examples of aforementioned natural carbohydrate are monosaccharide such as glucose, fructose etc; disaccharide such as maltose, sucrose etc; conventional sugar such as dextrin, cyclodextrin; and sugar alcohol such as xylitol, and erythritol etc. As the other deodorant than aforementioned ones, natural deodorant such as taumatin, stevia extract such as levaudioside A, glycyrrhizin et al., and synthetic deodorant such as saccharin, aspartam et al., may be useful favorably. The amount of above described natural carbohydrate is generally ranges from about 1 to 20 g, preferably 5 to 12 g in the ratio of 100 ml of present beverage composition.

The other components than aforementioned composition are various nutrients, a vitamin, a mineral or an electrolyte, synthetic flavoring agent, a coloring agent and improving agent in case of cheese chocolate et al., pectic acid and the salt thereof, alginic acid and the salt thereof, organic acid, protective colloidal adhesive, pH controlling agent, stabilizer, a preservative, glycerin, alcohol, carbonizing agent used in carbonate beverage et al. The other component than aforementioned ones may be fruit juice for preparing natural fruit juice, fruit juice beverage and vegetable beverage, wherein the component can be used independently or in combination. The ratio of the components is not so important but is generally range from about 0 to 20 w/w % per 100 w/w % present composition.

Examples of addable food comprising aforementioned crude drug composition therein are various food, beverage, gum, vitamin complex, health improving food and the like.

It will be apparent to those skilled in the art that various modifications and variations can be made in the compositions, use and preparations of the present invention without departing from the spirit or scope of the invention.

### ADVANTAGEOUS EFFECTS

The present invention is related to a composition comprising an extract of licorice or liquiritigenin isolated therefrom significantly decrease the blood concentration of LDH and ALT enzymes, central necrosis and inflammation in toxicant-induced hepato-toxicity animal model when the inventive extract or compound of the present invention was orally and intravenously administrated thereto. Accordingly, the inventive compositions according to the present invention are useful in the prevention and treatment of the liver diseases and can be used as safe and efficient hepato-protective.

### BRIEF DESCRIPTION OF THE DRAWINGS

The above and other objects, features and other advantages of the present invention will more clearly understood from the following detailed description taken in conjunction with the accompanying drawings, in which;

FIG. 1 shows the comparison between the hepato-protective effect of liquiritigenin (LQ) treatment group and DDB treatment group on the acetaminophen-induced liver injury of rats after 4-day's treatment;

FIG. 2 shows the comparison between the hepato-protective effect of each liquiritigenin (LQ) and DDB treatment group and the combination thereof on the acetaminophen-induced liver injury of rats after 4-day's treatment;

FIG. 3 shows the result of histochemical analysis in acetaminophen-induced liver injury of rats after 4-days treatment of the combination of liquiritigenin (LQ) and DDB (CV, PS, N, HD);

FIG. 4 represents the effect of liquiritigenin treatment on the concentration of ALT and LDH in acetaminophen-induced liver injury of rats after 2-day's treatment intravenously into tail vein;

FIG. 5 represents the result of histochemical analysis in acetaminophen-induced liver injury of rats after 2-days treatment of liquiritigenin (LQ) intravenously into tail vein (CV, N, HD, H);

FIGS. 6a and 6b present the result of histochemical analysis in acetaminophen-induced liver injury of GSH-depleted rats after 2-days treatment of liquiritigenin (LQ) orally (CV, PS, N, HD);

FIG. 7 present the result of histochemical analysis in acetaminophen-induced liver injury of rats after 3-days treatment of liquiritigenin (LQ) orally (CV, PS, N, HD).

### BEST MODE FOR CARRYING OUT THE INVENTION

It will be apparent to those skilled in the art that various modifications and variations can be made in the composition, use and preparations of the present invention without departing from the spirit or scope of the invention.

The present invention is more specifically explained by the following figures and examples. However, it should be understood that the present invention is not limited to these examples in any manner.

### MODE FOR THE INVENTION

The following Examples and Experimental Examples are intended to further illustrate the present invention without limiting its scope.

### Example 1

### Preparation of Inventive Extract of Licorice (EL)

3 kg of Glycyrrhiza uralensis purchased from Kyung-dong market located in Seoul were washed, dried and 15 liter of 100% methanol was added thereto. The solution was left alone for 72 hours at room temperature and the extract was filtered to obtain 290 g of crude extract of licorice (designated as EL hereinafter).

### Example 2

### Preparation of Inventive Liquiritigenin-Abundant Extract of Licorice (LEL)

290 g of extract (EL) prepared in Example 1 was subjected to Silicagel column chromatography (60 cm, 230-400 mesh) with eluting solution starting from CHCl₃ to mixture solvent (CHCl₃—MeOH=50:1→15:1) to obtain 43 g of purified fraction. The fraction was further subjected to Silicagel column chromatography (50 cm, 230-400 mesh) with eluting solution of mixture solvent (CHCl₃-acetone=20→11:1) to obtain 27 g of liquiritin-abundant fractions (designated as LAF hereinafter), i.e., fractions 32-67.

27 g of liquiritin-abundant fractions (LAP) was reacted with 1N HCl at 100° C. for 2 hours to hydrolyze the sugar-moiety of liquiritin and the reactant was neutralized with 1H NaOH. The solution was subjected to Silica gel column chromatography (50 cm, 230-400 mesh) eluting with mixture solvent system (CHCl₃ and acetone) to obtain 15 g of liquiritigenin-abundant extract of the present invention (designated as LEL hereinafter).

### Example 3

### Preparation of Inventive Liquiritigenin (LQ)

27 g of liquiritin-abundant fractions (LAF) prepared in Example 2 was further subjected to Silicagel column chromatography (50 cm, 230-400 mesh) with eluting solution of mixture solvent (CHCl₃-MeOH=50:1→15:1) to obtain 22 g of purified white powdered liquiritin.

22 g of liquiritin was reacted with 1N HCl at 100° C. for 2 hours to hydrolyze the sugar-moiety of liquiritin and the reactant was neutralized with 1H NaOH. The solution was subjected to Silica gel column chromatography (50 cm, 230-400 mesh) eluting with mixture solvent system (CHCl₃ and acetone) to obtain 12 g of liquiritigenin of the present invention (designated as LQ hereinafter).

### Example 4

### Preparation of Inventive Combinations

### 4-1. Preparation of Combination (C1)

The dried crude extract (EL) prepared in Example 1 was mixed with DDB (Pharmaking Pharmaceutical Co.) with the mixed ratio of 1:1, which was used in following experiments as a test sample (designated as C1 hereinafter).

### 4-2. Preparation of Combination (C2)

The dried liquiritigenin-abundant extract (LEL) prepared in Example 2 was mixed with DDB (Pharmaking Pharmaceutical Co.) with the mixed ratio of 1:1, which was used in following experiments as a test sample (designated as C2 hereinafter).

### 4-2. Preparation of Combination (C3)

The dried liquiritigenin (LQ) prepared in Example 3 was mixed with DDB (Pharmaking Pharmaceutical Co.) with the mixed ratio of 1:1, which was used in following experiments as a test sample (designated as C3 hereinafter).

### Reference Example

### Preparation of Experiment

### 1-1. Reagent and Experimental Animals

Acetaminophen (Sigma Chemical Co.) and BSO (Sigma Chemical Co.) were purchased from commercial company to use in experimental.

Acetaminophen was dissolved in 40% PEG solution (No. 400) in distilled water and DDB was dissolved in 0.5% methyl cellulose solution (No. 400) in distilled water

Male Sprague-Dawley rats (Samtako Co. Korea) weighing 140-160 g were used in the experiment and were allowed to access to feed (Harlan, teklan, USA) and drinking water. All animals were maintained in a controlled environment with temperatures at 22±2° C. and humidity at 55±5% with 12 hours of light and dark cycles for at least one week prior to use.

### 1-2. Statistics

All the result was analyzed by using pharmacological calculation. The significance between the test groups was evaluated by ANOVA (one-way analysis of variance) and determined by Newmann-Keuls test method (*p<0.5, **p<0.01).

### Experimental Example 1

### Effect of Orally-Administrated Inventive Extract and Compound on Acetaminophen-Induced Liver Injury in Rat Model

In order to investigate the inhibitory effect of the inventive extract and compound obtained in Examples on liver injury, following experiment was performed in the procedure.

### 1-1. Test Procedure

Liquiritigenin dissolved in 40% methycellulose solution in distilled water (25 mg.kg and 5 mg/kg) and DDB solution prepared in Reference example (50 mg/kg and 100 mg/kg) were orally administrated into the rats once a day for 4 days. 24 hours after the end of treatment, the acetaminophen solution prepared in Reference Example (1.2 g/kg) was orally administrated into the rats to induce hepatotoxicity.

24 hours after the treatment, the abdominal cavity was excised to deliver liver organ and blood to test the effect of the inventive extract and compound obtained in Examples on liver injury.

### 1-2. Test Result of the Indicators of Liver Injury

The effect on the indicators of liver injury known in the art, i.e., ALT (R. Rej, Clin. Chem., 24, pp 1971-1979, 1978) and LDH (S. Sherlock et al., Blackwell Science, London, p 23, 2002) activity in rat blood was determined.

### 1-2-1. The Effect of Liquritigenin and DDB

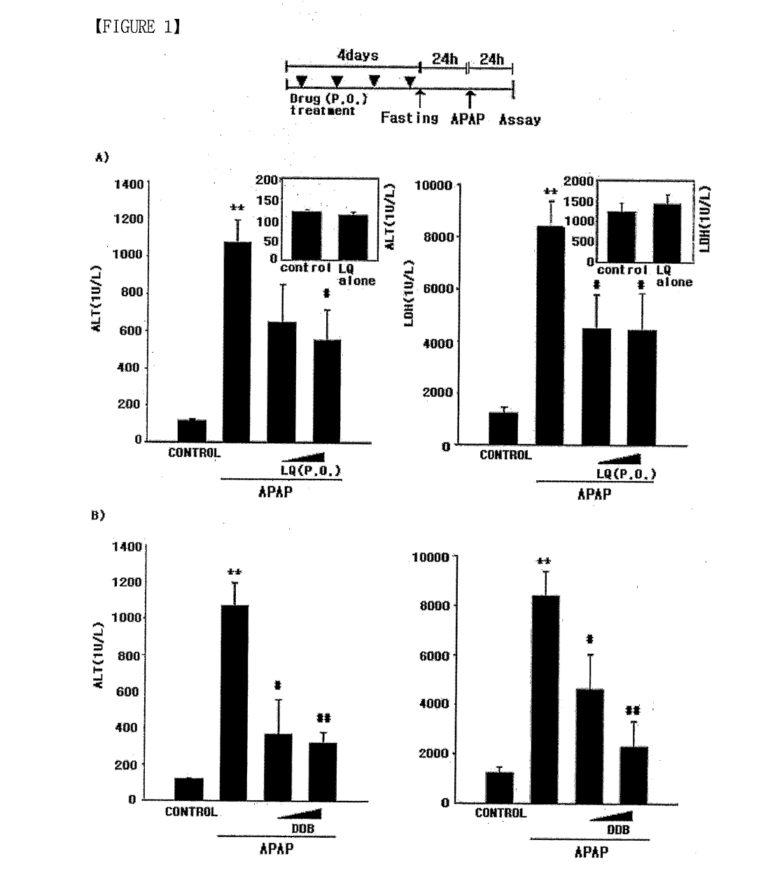

At the result, the increased activities of blood ALT and LDH enzymes induced by acetaminophen were significantly reduced by the treatment groups with liquiritigenin in oral dose of 25 mg/kg and 50 mg/kg (See FIG. 1) and the treatment groups of DDB in the dose of 50 mg/kg and 100 mg/kg also strikingly reduce the increased activities of blood ALT and LDH enzymes induced by acetaminophen.

### 1-2-2. The Effect of Combination of Liquritigenin and DDB

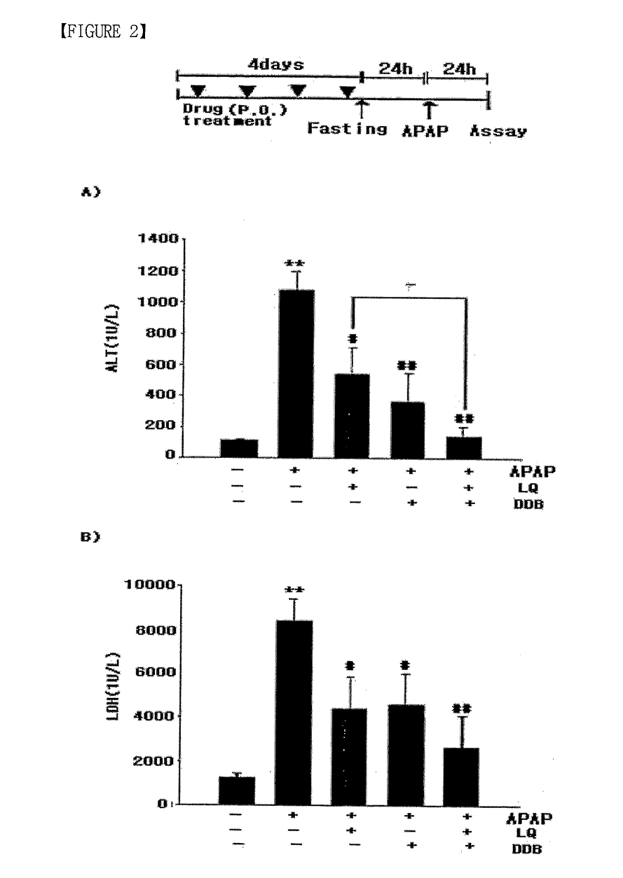

Combination of liquritigenin and DDB prepared in Example 4 was orally administrated into the rat at the dose of 50 mg/kg/day for 4 days. At the result, the combination of liquritigenin and DDB showed more decreasing effect on the increased activities of blood ALT and LDH enzymes induced by acetaminophen that respective group (See FIG. 2).

### 1-3. Test Result of Histo-Pathological Test

The histo-pathological examination on liver organ was performed to determine the effect of inventive extract and compound on the necrosis and inflammation of liver induced by acetaminophen.

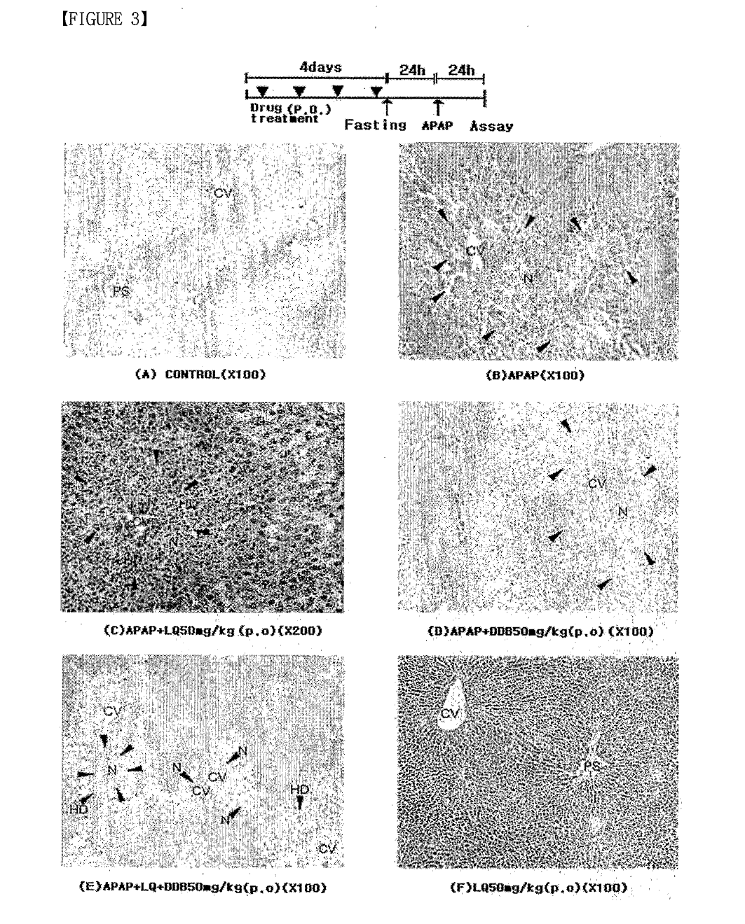

At the result, liquiritigenin strikingly reduced the central necrosis and inflammation of liver, which verify the direct preventive activity of liquritigenin. DDB inhibited the inflammation of liver induced by other toxic substances whereas it reduced little the central necrosis and inflammation of liver. The combination of liquritigenin and DDB showed similar reducing effect on the necrosis and inflammation of liver to respective group (ee FIG. 3 and Table 1).

TABLE 1

| | Central vein | Hepatic cell degeneration (HD) | | | | |
|---|---|---|---|---|---|---|
| Treatment* | neighboring | Central | Middle | Limbic | Inflammatory | Survival |
| (n = 10) | cell necrosis (N) | Vein (CV) | Region | region | cell | ratio (%) |
| Negative Control | 0 | 0 | 0 | 0 | 0 | 100 |
| APAP | 2.67 ± 0.7** | 0 | 0 | 0 | 0.67 ± 0.2** | 100 |
| APAP + LQ | 0.8 ± 0.8^{##} | 0.8 ± 0.8 | 0.6 ± 0.6 | 0.2 ± 0.2 | 0^{##} | 100 |
| (50 mg/kg, p.o) | | | | | | |
| APAP + DDB | 2.38 ± 0.5 | 0.4 ± 0.2 | 0.2 ± 0.2 | 0 | 0^{##} | 100 |
| (50 mg/kg, p.o) | | | | | | |
| APAP + LQ | 0.63 ± 0.7^{##} | 0.5 ± 0.3 | 0.25 ± 0.2 | 0.25 ± 0.3 | 0^{##} | 100 |
| (50 mg/kg, p.o) + DDB | | | | | | |
| (50 mg/kg, p.o) | | | | | | |
| APAP + LQ | 0.2 ± 0.2^{##} | 0.8 ± 0.4 | 0.6 ± 0.4 | 0 | 0 | 100 |
| (5 mg/kg, i. v) | | | | | | |
| APAP + BSO | 4**^{,##} | 0 | 0 | 3** | 0 | 26.7 |
| APAP + BSO + LQ | 2.88 ± 10.1^{††} | 1.5 ± 0.5 | 1.5 ± 0.5 | 1.5 ± 0.5^{††} | 0 | 60.0 |
| (50 mg/kg, p.o) | | | | | | |
| APAP + BSO + DDB | 3.88 ± 0.1 | 1.8 ± 0.2 | 1.8 ± 0.2 | 2 ± 0.02^{††} | 0 | 66.7 |
| (50 g/kg, p.o) | | | | | | |
| APAP + BSO + LQ | 2.83 ± 0.2^{††} | 2 | 2 | 2 ± 0.05^{††} | 0 | 73.3 |
| (50 mg/kg, p.o) + DDB | | | | | | |
| (50 mg/kg, p.o) | | | | | | |
| APAP + BSO + LQ | 2.83 ± 0.2^{††} | 0.2 ± 0.2 | 0.2 ± 0.2 | 2 ± 0.03^{††} | | 86.7 |
| (15 mg/kg,i.v.) | | | | | | |

*APAP(acetaminophen; LQ(liquiritigenin); BSO (buthionine sulfoximine).

### Experimental Example 2

### Effect of Intravenously-Administrated Inventive Extract and Compound on Acetaminophen-Induced Liver Injury in Rat Model

In order to investigate the inhibitory effect of the inventive extract and compound obtained in Examples on liver injury, following experiment was performed in the procedure.

### 2-1. Test Procedure

Liquiritigenin dissolved in 40% methycellulose solution in distilled water (5 mg.kg and 15 mg/kg) was intravenously administrated into the tail of rats. 3 hours after the end of treatment, the acetaminophen solution prepared in Reference Example (1.2 g/kg) was orally administrated into the rats to induce hepatotoxicity.

24 hours after the inducement, the abdominal cavity was excised to deliver liver organ and blood to test the effect of the inventive extract and compound obtained in Examples on liver injury.

### 2-2. Test Result

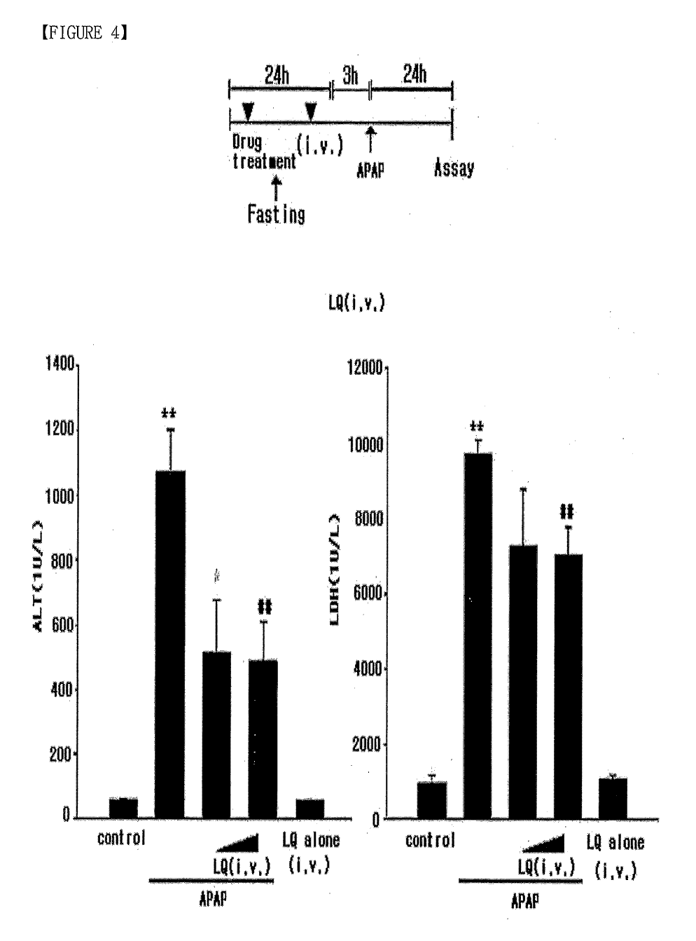

At the result, the increased activities of blood ALT and LDH enzymes induced by acetaminophen were significantly reduced by the treatment groups with liquiritigenin in intravenous dose of 5 mg/kg and 15 mg/kg (ee FIG. 4) and liquritigenin.

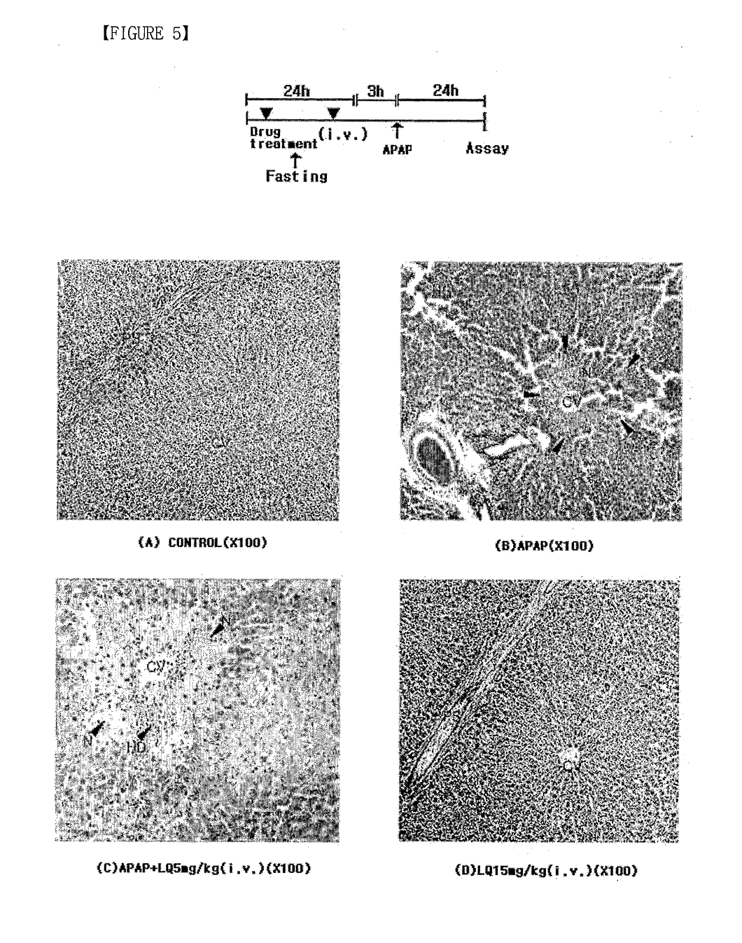

Intravenously administrated liquiritigenin strikingly reduced the central necrosis and inflammation of liver, which verifies that intravenous administration of liquiritigenin showed more potent than orally administration (See FIG. 5 and Table 1).

### Experimental Example 3

### Effect of Liquiritigenin on GSH-Depleted Liver Injury in Rat Model

In order to investigate the inhibitory effect of the inventive extract and compound obtained in Examples on liver injury, following experiment was performed in the procedure.

### 3-1. Test Procedure

Liquiritigenin solution (25 mg/kg and 50 mg/kg) and DDB solution (50 mg/kg and 100 mg/kg) was orally administrated into the tail rats a day for 2 days. 3 hours after the end of treatment, 2 mM/kg of BSO (buthionine sulfoximine) was intraperitoneally administrated to the rats to deplete GSH (glutathione) level of the rats. 1 hr after the treatment, the acetaminophen solution prepared in Reference Example (1.2 g/kg) was orally administrated into the rats to induce hepatotoxicity.

24 hours after the inducement, the survival rate of the rats was determined and the abdominal cavity was excised to deliver liver organ to test the effect of the inventive extract and compound obtained in Examples on liver injury.

### 3-2. Test Result

Based on the fact that the hepato-toxicity is deteriorated by the depletion of GSH level, present inventors tested the inhibitory effect of the treatment of sole liquiritigenin and combination with DDB on the GSH-depleted liver injury.

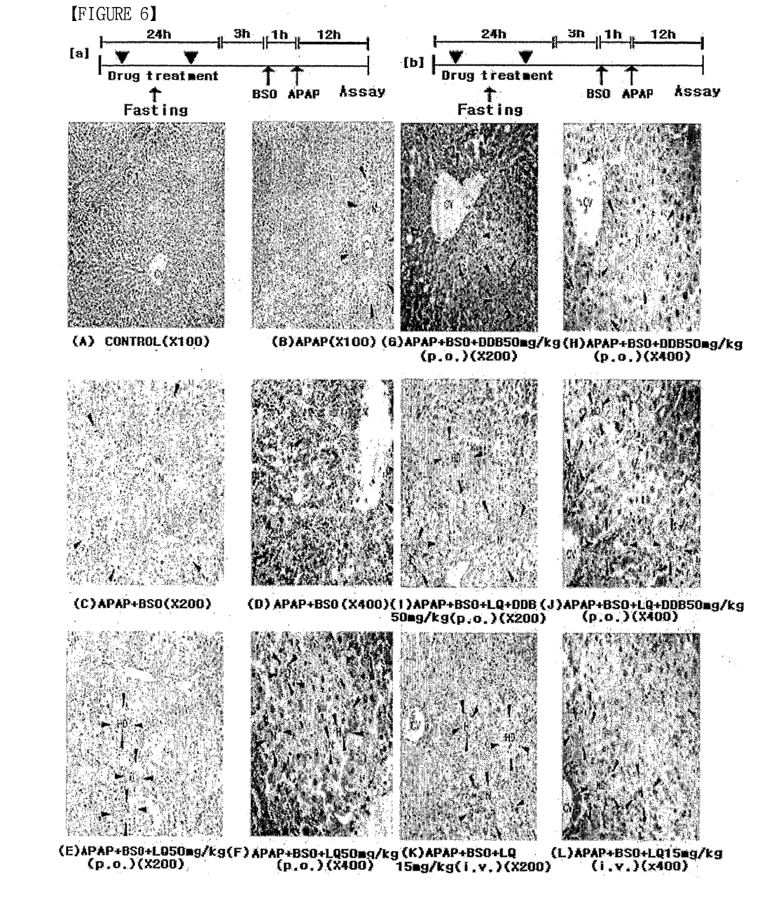

At the result, combined administration of acetaminophen and DDB showed extensive necrosis in hepatic cell (See FIG. 6a and Table 1). Orally administrated liquiritigenin strikingly reduced the necrosis of hepatic cell whereas DDB treatment could not reduce (See FIG. 6b). The combined administration of liquiritigenin and DDB potently inhibited the hepatic injury induced by acetaminophen (See FIG. 6b and Table 1), which verifies that combined administration of liquiritigenin and DDB still showed potent inhibitory effect on GSH-depleted liver injury. Additionally, intravenously administrated liquiritigenin for 2 days also apparently inhibits the hepatic injury (See FIG. 6b and Table 1).

After the observation of the survival rate, the combined treatment group more reduced the survival rate to 26.7% than control group while acetaminophen-treatment group did not show any change in the survival rate. Surprisingly, the survival rate in liquiritigenin-treatment group was strikingly increased and that in combination group of liquiritigenin and DDB was further increased as can be seen in Table 1.

### Experimental Example 4

### Treating in Order to Investigate the Treating Effect of the Inventive Extract and Compound Obtained in Examples on Liver Injury, Following Experiment was Performed in the Procedure

In order to investigate the treating effect of the inventive extract and compound obtained in Examples on liver injury, following experiment was performed in the procedure.

### 4-1. Test Procedure

The acetaminophen solution prepared in Reference Example (1.2 g/kg) was orally administrated into the rats to induce hepatotoxicity. 1 hour after the inducement, Liquiritigenin solution (50 mg/kg) was orally administrated into the rats once a day for 2 days. All the testing rats had been starved for 24 hours prior to acetaminophen-administration.

24 hours after the inducement, the abdominal cavity was excised to deliver liver organ to test the effect of the inventive compound obtained in Examples on liver injury.

### 4-2. Test Result

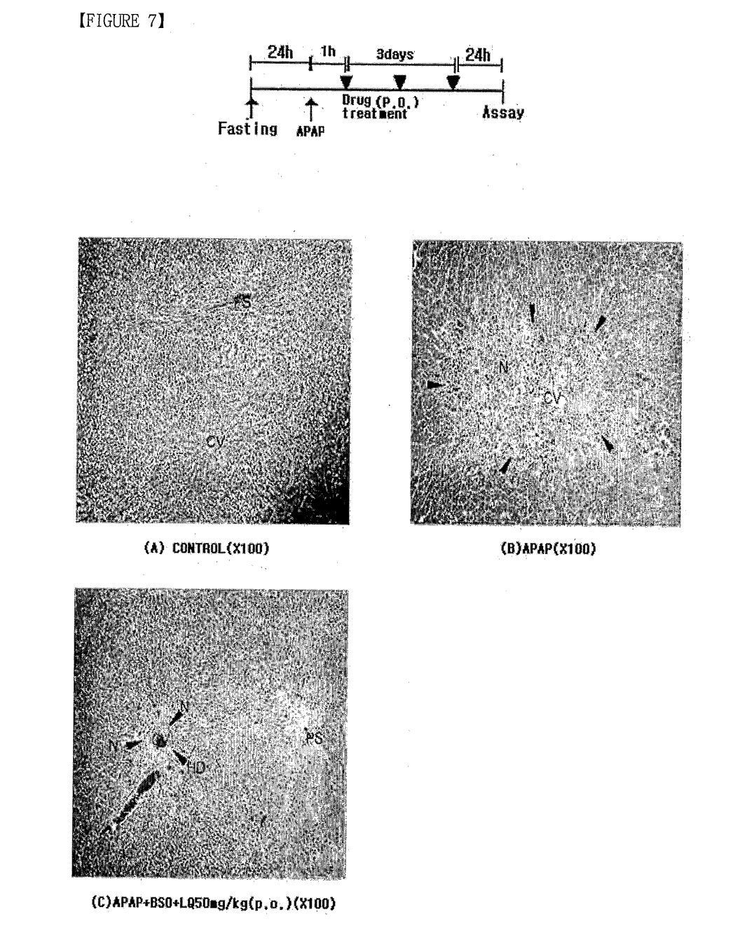

At the result, orally administrated liquiritigenin more strikingly inhibited the necrosis of hepatic cell than negative control group which had been treated with only PEG400 (40%) instead of liquiritigenin (ee FIG. 7), which verifies the directly treating effect of liquiritigenin on hepatic cell injury.

Hereinafter, the formulating methods and kinds of excipients will be described, but the present invention is not limited to them. The representative preparation examples were described as follows.

Preparation of injection

| | |
|---|---|
| Liquiritigenin (LQ) | 10 mg |
| Mannitol | 180 mg |
| Na₂HPO₄—12H₂O | 26 mg |
| Distilled water for injection | optimum amount |

Injection preparation was prepared by dissolving active component, controlling pH to about 7.5 and then filling all the components in 2 ml ample and sterilizing by conventional injection preparation method.

Preparation of powder

| | |
|---|---|
| liquiritigenin | 25 mg |
| DDB | 50 mg |
| Corn Starch | 20 mg |
| Lactose | 30 mg |
| Mg stearate | optimum amount |

Powder preparation was prepared by mixing above components and filling sealed package.

Preparation of tablet

| | |
|---|---|
| Liquiritigenin(LQ) | 100 mg |
| Corn Starch | 10 mg |
| Lactose | 50 mg |
| Magnesium stearate | optimum amount |

Tablet preparation was prepared by mixing above components and entabletting.

Preparation of capsule

| | |
|---|---|
| Liquiritigenin | 25 mg |
| DDB | 50 mg |
| Lactose | 50 mg |
| Corn starch | 28 mg |
| Talc | 2 mg |
| Magnesium stearate | optimum amount |

Tablet preparation was prepared by mixing above components and filling gelatin capsule by conventional gelatin preparation method.

Preparation of soft capsule

| | |
|---|---|
| Liquiritigenin | 500 mg |
| Polyethylene glycol 400 | 400 mg |
| Conc-glycerin | 55 mg |
| Distilled water | 35 mg |

PEG and conc-glycerin were mixed together and then distilled water was added thereto. The mixture was stirred to mix homogeneously at the speed of about 1500 rpm at 60° C. and then the mixture was cooled at room temperature to use as the content of capsule. The coating of the capsule was prepared by conventional preparation method for example, all the component, i.e., 132 mg of gelatin, 52 mg of conc-glycerin, 6 mg of 70% di-sorbitol solution, coloring agent such as ethyl vanillin, and coating matrix such as carnauba Pb were mixed together to make soft capsule.

Preparation of Suspension

| Liquiritigenin (LQ) | 500 | mg |
|---|---|---|
| High maltose syrup | 10 | g |
| Sugar | 30 | mg |
| CMC Na | 100 | mg |
| Lemon flavor | appropriate amount | |
| Distilled water | 100 | ml |

Suspension preparation was prepared by conventional preparation method known in the art. The components was filled in 1000 ml brown color bottle to sterilize by conventional liquid preparation method.

Preparation of liquid

| | | |
|---|---|---|
| Liquiritigenin(LQ) | 1000 | mg |
| Sugar | 20 | g |
| Polysaccharide | 20 | g |
| Lemon flavor | 20 | g |

Liquid preparation was prepared by dissolving active component, and then filling all the components in 1000 ml ample and sterilizing by conventional liquid preparation method.

Preparation of health food

| Liquiritigenin(LQ) | 1000 | mg |
|---|---|---|
| Vitamin mixture | optimum amount | |
| Vitamin A acetate | 70 | μg |
| Vitamin E | 1.0 | mg |
| Vitamin B₁ | 0.13 | mg |
| Vitamin B₂ | 0.15 | mg |
| Vitamin B₆ | 0.5 | mg |
| Vitamin B₁₂ | 0.2 | μg |
| Vitamin C | 10 | mg |
| Biotin | 10 | μg |
| Amide nicotinic acid | 1.7 | mg |
| Folic acid | 50 | μg |
| Calcium pantothenic acid | 0.5 | mg |
| Mineral mixture | optimum amount | |
| Ferrous sulfate | 1.75 | mg |
| Zinc oxide | 0.82 | mg |
| Magnesium carbonate | 25.3 | mg |
| Monopotassium phosphate | 15 | mg |
| Dicalcium phosphate | 55 | mg |
| Potassium citrate | 90 | mg |
| Calcium carbonate | 100 | mg |
| Magnesium chloride | 24.8 | mg |

The above mentioned vitamin and mineral mixture may be varied in many ways. Such variations are not to be regarded as a departure from the spirit and scope of the present invention.

Preparation of health beverage

| | | |
|---|---|---|
| Liquiritigenin(LQ) | 1000 | mg |
| Citric acid | 1000 | mg |
| Oligosaccharide | 100 | g |
| Apricot concentration | 2 | g |
| Taurine | 1 | g |
| Distilled water | 900 | ml |

Health beverage preparation was prepared by dissolving active component, mixing, stirred at 85° C. for 1 hour, filtered and then filling all the components in 1000 ml ample and sterilizing by conventional health beverage preparation method.

The invention being thus described, it will be obvious that the same may be varied in many ways. Such variations are not to be regarded as a departure from the spirit and scope of the present invention, and all such modifications as would be obvious to one skilled in the art are intended to be included within the scope of the following claims.

### INDUSTRIAL APPLICABILITY

As described in the present invention, the inventive extract or compound significantly decrease the blood concentration of LDH and ALT enzyme, central necrosis and inflammation in acetaminophen-induced hepato-toxicity animal model when the inventive extract or compound of the present invention was orally and intravenously administrated thereto. The inventive compositions according to the present invention are useful in the prevention and treatment of the liver diseases and can be used as safe and efficient hepato-protective agent.

1. A pharmaceutical composition comprising the extract of licorice or liquiritigenin isolated therefrom as an active ingredient in an amount effective to preventing and treating liver disease, together with a pharmaceutically acceptable carrier.

2. The pharmaceutical composition according to claim 1, wherein said licorice is glycyrrhiza uralensis or glycyrrhiza glabra L.

3. The pharmaceutical composition according to claim 1, wherein said extract is extracted with distilled water, lower alcohols, or the mixtures thereof.

4. The pharmaceutical composition according to claim 1, wherein said extract is liquiritigenin-abundant extract of licorice, which is prepared by the procedure consisting of the steps: purifying the extract of licorice with repeated column chromatographic method to obtain purified liquiritin fraction; treating to acidic hydrolysis and neutralizing the pH of the solution with alkali; and subjecting column chromatography to obtain the purposed liquiritigenin-abundant extract.

5. The pharmaceutical composition according to claim 1, wherein said extract or liquiritigenin further comprises DDB(dimethyl-4,4′-dimethoxy-5,6,5′,6′-dimethylene dioxybiphenyl-2,2′-dicarboxylate).

6. The pharmaceutical composition according to claim 1, wherein said liver disease is acute or chronic hepatitis, hepatomegaly, hepatophyma, hepatocirrhosis or liver cancer.

7. A use of extract of licorice or liquiritigenin isolated therefrom for manufacture of medicament employed for treating or preventing liver disease in human or mammal.

8. A method for treating liver diseases by protecting hepatic cell in a mammal comprising administering to said mammal an effective amount of extract of licorice or liquiritigenin isolated therefrom, together with a pharmaceutically acceptable carrier thereof.

9. A method for preparing liquiritigenin-abundant extract from the extract of licorice comprising the steps consisting of; washing, drying licorice; mixing with 5 to 15-fold volume of distilled water, alcohols such as methanol, ethanol and the like, or the mixtures thereof; enfleuraging the solution at the temperature ranging from 0 to room temperature, 48 to 72 hours; filtering the residue to obtain the crude extract of licorice; subjecting the extract to repeated column chromatography eluted with mixed solvent system to obtain liquiritin-abundant extract of licorice; subjecting the extract to acid hydrolysis using by strong acid to remove the sugar-moiety of liquiritin; neutralizing the solution with alkali solution; subjecting the solution to Silica gel column chromatography eluting with mixture solvent system (CHCl₃ and acetone) to obtain liquiritigenin-abundant extract of the present invention.

10. A health functional food comprising an extract of licorice or liquiritigenin isolated therefrom for the prevention or improvement of liver disease by protecting hepatic cell as an active ingredient in an amount effective to preventing and improving liver disease, together with a sitologically acceptable additive.

11. A health functional food according to claim 9 said extract or liquiritigenin further comprises DDB (dimethyl-4,4′-dimethoxy-5,6,5′,6′-dimethylene dioxybiphenyl-2,2′-dicarboxylate).
